(19) United States
(12) Patent Application Publication
(10) Pub. No.: US 2024/0216255 A1
(43) Pub. Date: Jul. 4, 2024

(54) HYDROGELS INCLUDING BIOPOLYMERS

(71) Applicant: BEIERSDORF AG, Hamburg (DE)

(72) Inventors: Dana BOELKE, Hamburg (DE); Celina VORPAHL, Hamburg (DE)

(21) Appl. No.: 18/556,904

(22) PCT Filed: May 24, 2022
(86) PCT No.: PCT/EP2022/064073

§ 371 Date: Oct. 24, 2023

(30) Foreign Application Priority Data
May 25, 2021 (DE) 10 2021 205 299.1
Aug. 17, 2021 (DE) 10 2021 209 006.0

(51) Int. Cl. A61K 8/73; A61K 8/04; A61Q 19/10

(52) U.S. Cl. CPC A61K 8/73; A61K 8/042; A61Q 19/10; A61K 2800/594

(57) ABSTRACT

The present invention relates to a cosmetic hydrogel containing dehydroxanthan gum and xanthan gum and/or carrageenan, and to the use of xanthan gum and/or carrageenan for viscosity stabilization of a hydrogel comprising dehydroxanthan gum.

Cosmetic products generally not only serve to appear beautiful and attractive, but their effects make a decisive contribution to increased self-esteem and people's wellbeing. Accordingly, a wide variety of cosmetic products are used for daily cleansing and care of human skin.

The present invention relates to a water-containing transparent gel (hydrogel), which comprises at least two gel formers in addition to water. Preferably, the gel according to the invention comprises at least one cosmetic active ingredient.

Hydrogels and use thereof for administering cosmetic ingredients to a person's skin or mucous membrane are known. In a technical sense, the term “gel” is understood to mean relatively dimensionally stable, readily deformable disperse systems composed of at least two components, which usually consist of a—mostly solid—colloidal divided substance composed of long-chain molecular groupings (e.g., gelatin, silica, polysaccharides) as structure former and a liquid dispersant (for example water). The colloidal divided substance is often referred to as a thickener, gel former or gelling agent. It forms a spatial network in the dispersant, whereby individual colloidal particles can be more or less firmly linked to one other via electrostatic interaction.

The thickeners used are mainly acrylic acid polymers. The free carboxyl groups present in these polymers are converted to the salt form by alkalis—such as aqueous sodium hydroxide or amines—and thus result in an increase in viscosity as a result of strong crosslinking.

Cellulose derivatives or other polysaccharides, such as guar flour or xanthan gum, are also used as gel formers. Depending on the type and amount of thickener, the consistency of a gel can be adjusted from liquid to cut-resistant.

The dispersant which surrounds the network is characterized by electrostatic affinity to the thickener, i.e., a predominantly polar (in particular: hydrophilic) thickener preferably gelates a polar dispersant (in particular: water), whereas a predominantly non-polar thickener preferably gelates non-polar dispersants.

Strong electrostatic interactions, which are realized, for example, in hydrogen bonds between thickener and dispersant, but also between dispersant molecules themselves, can also lead to strong crosslinking of the dispersant. Hydrogels may consist of almost 100% water (in addition to, for example, ca. 0.2-1.0% of a gelling agent) and have quite a firm consistency.

In cosmetic galenics, gels are generally characterized by a semi-solid, often free-flowing consistency.

The person skilled in the art knows hydrogels from DE2005056497 A1, inter alia.

It is known in the prior art that acrylate-based polymers are often used for gel formation. Acrylate-based polymers are polymers that are obtained from homo- or copolymerization with acrylic and/or methacrylic acid. Examples include, inter alia, sodium polyacrylates or acrylate copolymers. As an alternative to acrylate-based polymers, it is also possible to use polymers formed by homo- or copolymerization with vinyl pyrrolidone for gel formation.

A disadvantage, however, is that the use of these aforementioned polymers is increasingly being criticized as their biodegradability has not been fully clarified. Consequently, there is a need for hydrogels that are not based on the use of these polymers.

However, there are unforeseen problems in the provision of transparent hydrogels. For instance, the viscosity of the hydrogels often increases continuously during storage, so that the properties of the hydrogels change depending on the storage time.

Surprisingly, it has now been found that the disadvantages of the prior art could be eliminated or alleviated by the present invention.

Subject matter of the present invention is a cosmetic hydrogel having
a) dehydroxanthan gum and
b) xanthan gum and/or carrageenan and
c) water.

A further subject matter of the invention is the use of xanthan gum and/or carrageenan for viscosity stabilization of a hydrogel having dehydroxanthan gum and water.

If percentages by weight (% by weight) are given below without reference to a particular composition or specific mixture, then these figures always refer to the total weight of the hydrogel. If ratios of components/substances/substance groups are disclosed below, these ratios refer to ratios by weight of the components/substances/substance groups specified.

If ranges of percentages by weight are given below for the constituents of the hydrogel, the disclosure of the present application also includes all individual values in steps of 0.1% by weight within these weight percentage ranges.

The expressions “according to the invention”, “advantageous according to the invention”, “advantageous in the context of the present invention” etc. always relate in the context of the present disclosure to both the hydrogel according to the invention and to the use according to the invention.

All experiments were carried out under standard conditions unless stated otherwise. The expression “standard conditions” signifies 20° C., 1013 hPa and a relative humidity of 50%.

If the term skin is used, this preferably refers to human skin.

Emulsifiers are understood to mean all substances which are listed in the International Cosmetic Ingredient Dictionary and Handbook, Thirteenth Edition 2010, (ISBN 1-882621-47-6) under the designation “emulsifying agent”. Surfactants are understood to mean all substances which are listed in the International Cosmetic Ingredient Dictionary and Handbook, Thirteenth Edition 2010, (ISBN 1-882621-47-6) under the designation “surfactant”.

Where viscosity values are given in this disclosure, all values refer to a measurement at 25° C. in a 150 ml wide-neck bottle (VWR No.: 807-001) using Rheomat R 123 from proRheo. The Rheomat R 123 from proRheo GmbH is a rotational viscometer, i.e., a measurement body rotates in the substance to be measured. The force is measured that is required to rotate the measurement body in the sample at a predefined speed. The viscosity is calculated from this torque, the speed of the measurement body and the geometric dimensions of the measuring system used. The measurement body used is measurement body No. 1 (article no. 200 0191), suitable for a viscosity range up to 10 000 [mPa·s], rotational speed range 62.5 min-1.

According to the invention, the cosmetic hydrogel comprises dehydroxanthan gum. It is advantageous in the context of the present invention if the proportion of dehydroxanthan gum is from 0.1% by weight to 5% by weight, preferably from 0.3% by weight to 3.5% by weight and particularly preferably from 0.6% by weight to 2% by weight, based on the total weight of the hydrogel.

Furthermore, in accordance with the invention, xanthan gum and/or carrageenan are present. Advantageously, the total proportion of xanthan gum and/or carrageenan is from 0.1 to 3% by weight, preferably from 0.15% by weight to 1.5% by weight, and particularly preferably from 0.2 to 0.7% by weight, based on the total weight of the hydrogel.

If xanthan gum is present, the proportion of xanthan gum is advantageously from 0.1 to 3% by weight, preferably from 0.15% by weight to 1.5% by weight, and particularly preferably from 0.2 to 0.7% by weight, based on the total weight of the hydrogel.

If carrageenan is present, the proportion of carrageenan is advantageously from 0.1 to 3% by weight, preferably from 0.15% by weight to 1.5% by weight, and particularly preferably from 0.2 to 0.7% by weight, based on the total weight of the hydrogel.

The ratio by weight of dehydroxanthan gum to the total content of xanthan gum and/or carrageenan is advantageously from 10:1 to 1:1, preferably from 8:1 to 1.5:1 and particularly preferably from 5:1 to 2:1.

The total proportion of water is advantageously at least 70% by weight, preferably at least 75% by weight and particularly preferably at least 77% by weight, based on the total weight of the hydrogel. The total proportion of water is advantageously less than 99% by weight, based on the total weight of the hydrogel.

The hydrogel according to the invention is preferably free from polymers obtained from homo- or copolymerization with vinyl pyrrolidone, acrylic acid and/or methacrylic acid. In the context of the present invention “free from” signifies that the total proportion of said substances is less than 0.05% by weight and particularly preferably 0% by weight, the figures referring to the total weight of the hydrogel.

It is also advantageous if the hydrogel is free from emulsifiers and surfactants.

Furthermore, it is advantageous in accordance with the invention if the hydrogel comprises further ingredients which are commonly used in such formulations.

Accordingly, it is advantageous if the hydrogel comprises one or more short-chain polyols having 2 to 8 carbon atoms. Advantageously, the proportion of these polyols having 2 to 8 carbon atoms is from 1 to 30% by weight, preferably from 2 to 23% by weight and particularly preferably from 3 to 20% by weight, based on the total weight of the hydrogel.

Preferred polyols having 2 to 8 carbon atoms are selected from the group consisting of glycerin, methylpropanediol, propylene glycol, hexane-1,2-diol, caprylyl glycol, butylene glycol and pentylene glycol.

If the hydrogel comprises glycerin, it is preferred when the proportion of glycerin is from 5 to 15% by weight, preferably from 7 to 12.5 and particularly preferably from 8 to 12% by weight, based on the total weight of the hydrogel.

If the hydrogel comprises methylpropanediol, it is preferred when the proportion of methylpropanediol is from 1 to 10% by weight, preferably from 2 to 8 and particularly preferably from 3 to 5% by weight, based on the total weight of the hydrogel.

If the hydrogel comprises propylene glycol, it is preferred when the proportion of propylene glycol is from 1 to 10% by weight, preferably from 2 to 8 and particularly preferably from 3 to 5% by weight, based on the total weight of the hydrogel.

If the hydrogel comprises hexane-1,2-diol, it is preferred when the proportion of hexane-1,2-diol is from 0.2 to 2% by weight, preferably from 0.5 to 1.5 and particularly preferably from 0.7 to 1.3% by weight, based on the total weight of the hydrogel.

It is also advantageous if the hydrogel comprises phenoxyethanol, wherein it is advantageous if the proportion of phenoxyethanol is from 0.2 to 1.5% by weight, preferably from 0.4 to 1.0% by weight and particularly preferably from 0.5 to 0.9% by weight, based on the total weight of the hydrogel.

Furthermore, it is advantageous according to the invention if further active ingredients are present. These are advantageously selected from the group sodium hyaluronate, 1-methylhydantoin-2-imide, creatine, folic acid, panthenol, pantolactone, vitamin C, magnolia, calcium pantothenate and sodium ascorbyl phosphate. The active ingredients cited in the group are advantageously used in total proportions of up to 2% by weight, based on the total weight of the hydrogel.

In addition, further cosmetic auxiliaries, such as other active ingredients, dyes, rheology modifiers, alcohols, oils and waxes may be present, provided they do not adversely affect the properties according to the invention.

Advantageously, the gels of the invention are characterized in that they are transparent. This property is often valued by consumers. A gel is considered transparent if it shows a transmission at 500 nm of 85% under standard conditions.

### COMPARATIVE EXPERIMENTS AND EXAMPLES

The examples below are intended to illustrate the present invention without limiting it. Unless otherwise stated, all quantitative data, fractions, and percentages are based on the weight and the total amount or on the total weight of the preparations.

The following tables show different compositions of hydrogels, and also the measured viscosity after different storage periods under standard conditions. Viscosity was measured as stated in the description above. Ex. 1 to Ex. 4 are comparative examples. Ex. 5 to Ex. 8 are according to the invention.

| Ingredients | Ex. 1 | Ex. 2 | Ex. 3 | Ex. 4 | Ex. 5 |
|---|---|---|---|---|---|
| Dehydroxanthan gum | 0.7 | 0.8 | 0.9 | 1.0 | 0.7 |
| Carrageenan | | | | | 0.3 |
| Xanthan gum | | | | | |
| Aqua | To | To | To | To | To |
| | 100 | 100 | 100 | 100 | 100 |
| Storage time and viscosity | | | | | |
| data in mPa · s: | | | | | |
| 1 d | 1400 | 2000 | 2950 | 3600 | 3650 |
| 7 d | 2050 | 2700 | 3500 | 4250 | Not |
| | | | | | measured |
| 30 d | 2500 | 3250 | 3950 | 4750 | 4200 |
| 60 d | 2650 | 3400 | 4000 | 4850 | 3850 |
| 120 d | 2850 | 3500 | 3950 | 4800 | 4350 |

| Ingredients | Bsp. 6 | Bsp. 7 | Bsp. 8 |
|---|---|---|---|
| Dehydroxanthan | 1.0 | 1.4 | 0.7 |
| gum | | | |
| Carrageenan | 0.3 | 0.3 | |
| Xanthan gum | | | 0.3 |
| Aqua | To 100 | To 100 | To 100 |
| Storage time | | | |
| and viscosity | | | |
| data in mPa · s: | | | |
| 1d | Not measured | 4900 | 2700 |
| 7d | 3250 | 4750 | 2900 |
| 30d | 3300 | 4750 | 3150 |
| 60d | 3200 | 4700 | 2950 |
| 120d | 3450 | 4700 | 3200 |

A comparison shows that, on storage up to 120 days, the increase in viscosity could be reduced by adding carrageenan or xanthan gum. Thus, more viscosity-stable hydrogels could be provided. The hydrogels according to the invention appear transparent.

Further examples according to the invention are shown below:

TABLE 1

| | Formulation | Formulation | Formulation | Formulation | Formulation | Formulation |
|---|---|---|---|---|---|---|
| Ingredients | 1 | 2 | 3 | 4 | 5 | 6 |
| Sodium hyaluronate | 0.225 | 0.185 | 0.195 | 0.175 | 0.18 | 0.19 |
| Hexane-1,2-diol | 1 | 1.2 | 1 | 0.9 | 1 | 1.35 |
| Methylpropanediol | 4.35 | 4 | 4.5 | 4 | 4.2 | 4 |
| Propylene glycol | 4.6 | 4.2 | 4.6 | 5 | 4.6 | 3.6 |
| Glycerin | 8 | 10 | 11 | 10 | 12.5 | 10 |
| Phenoxyethanol | 0.8 | 0.82 | 0.8 | 0.75 | 0.8 | 0.9 |
| Carrageenan | 0.35 | 0.3 | 0.32 | 0.3 | 0.26 | 0.3 |
| Dehydroxanthan gum | 1.4 | 1.2 | 1.4 | 0.95 | 1.4 | 1.5 |
| 1-methylhydantoin- | 0.03 | | | | | |
| 2-imide | | | | | | |
| Creatine | 0.15 | | | | | |
| Folic acid | | 0.12 | | | | |
| Panthenol + | | | 0.55 | | | |
| pantolactone + citric acid | | | | | | |
| Magnolia bark extract | | | | 0.07 | | |
| Ascorbic acid | | | | | | 0.5 |
| Calcium pantothenate | | | | | | |
| Sodium ascorbyl | | | | | 0.45 | |
| phosphate | | | | | | |
| Aqua | to 100 | to 100 | to 100 | to 100 | to 100 | to 100 |

TABLE 2

| | Formu- | Formu- | Formu- | Formu- | Formu- |
|---|---|---|---|---|---|
| Ingredients | lation 7 | lation 8 | lation 9 | lation 10 | lation 11 |
| Sodium hyaluronate | 0.21 | 0.22 | 0.25 | 0.15 | |
| Hexane-1,2-diol | 1 | 0.8 | 1 | 0.75 | 1 |
| Methylpropanediol | 3.5 | 4 | 3.7 | 4 | 3.85 |
| Propylene glycol | 4.6 | 3.95 | 4.6 | 4.75 | 4.6 |
| Glycerin | 9.5 | 10 | 7.5 | 10 | 10.75 |
| Phenoxyethanol | 0.8 | 0.92 | 0.8 | 0.85 | 0.8 |
| Carrageenan | 0.2 | 0.3 | 0.28 | 0.3 | 0.4 |
| Dehydroxanthan gum | 1.4 | 1.65 | 1.4 | 0.8 | 1.15 |
| 1-methylhydantoin- | | | | | |
| 2-imide | | | | | |
| Creatine | | | | | |
| Folic acid | | | | | |
| Panthenol + | | | | | |
| pantolactone + | | | | | |
| citric acid | | | | | |
| Magnolia bark extract | | | | | |
| Ascorbic acid | | | | | |
| Calcium pantothenate | 0.05 | | | | |
| Sodium ascorbyl | | | | | |
| phosphate | | | | | |
| Aqua | to 100 | to 100 | to 100 | to 100 | to 100 |

TABLE 3

| | Formulation | Formulation | Formulation | Formulation | Formulation | Formulation |
|---|---|---|---|---|---|---|
| Ingredients | 1 | 2 | 3 | 4 | 5 | 6 |
| Sodium hyaluronate | 0.225 | 0.185 | 0.195 | 0.175 | 0.18 | 0.19 |
| Hexane-1,2-diol | 1 | 1.2 | 1 | 0.9 | 1 | 1.35 |
| Methylpropanediol | 4.35 | 4 | 4.5 | 4 | 4.2 | 4 |
| Propylene glycol | 4.6 | 4.2 | 4.6 | 5 | 4.6 | 3.6 |
| Glycerin | 8 | 10 | 11 | 10 | 12.5 | 10 |
| Phenoxyethanol | 0.8 | 0.82 | 0.8 | 0.75 | 0.8 | 0.9 |
| Xanthan gum | 0.35 | 0.3 | 0.32 | 0.3 | 0.26 | 0.3 |
| Dehydroxanthan gum | 1.4 | 1.2 | 1.4 | 0.95 | 1.4 | 1.5 |
| 1-methylhydantoin-2- | 0.03 | | | | | |
| imide | | | | | | |
| Creatine | 0.15 | | | | | |
| Folic acid | | 0.12 | | | | |
| Panthenol + | | | 0.55 | | | |
| pantolactone + citric acid | | | | | | |
| Magnolia bark extract | | | | 0.07 | | |
| Ascorbic acid | | | | | | 0.5 |
| Calcium pantothenate | | | | | | |
| Sodium ascorbyl | | | | | 0.45 | |
| phosphate | | | | | | |
| Aqua | to 100 | to 100 | to 100 | to 100 | to 100 | to 100 |

TABLE 4

| | Formu- | Formu- | Formu- | Formu- | Formu- |
|---|---|---|---|---|---|
| Ingredients | lation 7 | lation 8 | lation 9 | lation 10 | lation 11 |
| Sodium hyaluronate | 0.21 | 0.22 | 0.25 | 0.15 | |
| Hexane-1,2-diol | 1 | 0.8 | 1 | 0.75 | 1 |
| Methylpropanediol | 3.5 | 4 | 3.7 | 4 | 3.85 |
| Propylene glycol | 4.6 | 3.95 | 4.6 | 4.75 | 4.6 |
| Glycerin | 9.5 | 10 | 7.5 | 10 | 10.75 |
| Phenoxyethanol | 0.8 | 0.92 | 0.8 | 0.85 | 0.8 |
| Xanthan gum | 0.2 | 0.3 | 0.28 | 0.3 | 0.4 |
| Dehydroxanthan gum | 1.4 | 1.65 | 1.4 | 0.8 | 1.15 |
| 1-methylhydantoin- | | | | | |
| 2-imide | | | | | |
| Creatine | | | | | |
| Folic acid | | | | | |
| Panthenol + | | | | | |
| pantolactone + | | | | | |
| citric acid | | | | | |
| Magnolia bark extract | | | | | |
| Ascorbic acid | | | | | |
| Calcium pantothenate | 0.05 | | | | |
| Sodium ascorbyl | | | | | |
| phosphate | | | | | |
| Aqua | to 100 | to 100 | to 100 | to 100 | to 100 |

TABLE 5

| | Formulation | Formulation | Formulation | Formulation | Formulation | Formulation |
|---|---|---|---|---|---|---|
| Ingredients | 1 | 2 | 3 | 4 | 5 | 6 |
| Sodium hyaluronate | 0.225 | 0.185 | 0.195 | 0.175 | 0.18 | 0.19 |
| Hexane-1,2-diol | 1 | 1.2 | 1 | 0.9 | 1 | 1.35 |
| Methylpropanediol | 4.35 | 4 | 4.5 | 4 | 4.2 | 4 |
| Propylene glycol | 4.6 | 4.2 | 4.6 | 5 | 4.6 | 3.6 |
| Glycerin | 8 | 10 | 11 | 10 | 12.5 | 10 |
| Phenoxyethanol | 0.8 | 0.82 | 0.8 | 0.75 | 0.8 | 0.9 |
| Xanthan gum | 0.15 | 0.15 | 0.17 | 0.15 | 0.13 | 0.3 |
| Carrageenan | 0.2 | 0.15 | 0.15 | 0.15 | 0.1 | 0.1 |
| Dehydroxanthan gum | 1.4 | 1.2 | 1.4 | 0.95 | 1.4 | 1.5 |
| 1-methylhydantoin-2- | 0.03 | | | | | |
| imide | | | | | | |
| Creatine | 0.15 | | | | | |
| Folic acid | | 0.12 | | | | |
| Panthenol + | | | 0.55 | | | |
| pantolactone + citric acid | | | | | | |
| Magnolia bark extract | | | | 0.07 | | |
| Ascorbic acid | | | | | | 0.5 |
| Calcium pantothenate | | | | | | |
| Sodium ascorbyl | | | | | 0.45 | |
| phosphate | | | | | | |
| Aqua | to 100 | to 100 | to 100 | to 100 | to 100 | to 100 |

TABLE 6

| | Formu- | Formu- | Formu- | Formu- | Formu- |
|---|---|---|---|---|---|
| Ingredients | lation 7 | lation 8 | lation 9 | lation 10 | lation 11 |
| Sodium hyaluronate | 0.21 | 0.22 | 0.25 | 0.15 | |
| Hexane-1,2-diol | 1 | 0.8 | 1 | 0.75 | 1 |
| Methylpropanediol | 3.5 | 4 | 3.7 | 4 | 3.85 |
| Propylene glycol | 4.6 | 3.95 | 4.6 | 4.75 | 4.6 |
| Glycerin | 9.5 | 10 | 7.5 | 10 | 10.75 |
| Phenoxyethanol | 0.8 | 0.92 | 0.8 | 0.85 | 0.8 |
| Xanthan gum | 0.2 | 0.3 | 0.28 | 0.1 | 0.1 |
| Carrageenan | 0.2 | 0.1 | 0.15 | 0.25 | 0.3 |
| Dehydroxanthan gum | 1.4 | 1.65 | 1.4 | 0.8 | 1.15 |
| 1-methylhydantoin- | | | | | |
| 2-imide | | | | | |
| Creatine | | | | | |
| Folic acid | | | | | |
| Panthenol + | | | | | |
| pantolactone + | | | | | |
| citric acid | | | | | |
| Magnolia bark extract | | | | | |
| Ascorbic acid | | | | | |
| Calcium pantothenate | 0.05 | | | | |
| Sodium ascorbyl | | | | | |
| phosphate | | | | | |
| Aqua | to 100 | to 100 | to 100 | to 100 | to 100 |

1.-14. (canceled)

15. A cosmetic hydrogel, wherein the hydrogel comprises
a. dehydroxanthan gum;
b. xanthan gum and/or carrageenan; and
c. water.

16. The cosmetic hydrogel of claim 15, wherein the hydrogel comprises from 0.1% to 5% by weight of dehydroxanthan gum, based on a total weight of the hydrogel.

17. The cosmetic hydrogel of claim 15, wherein the hydrogel comprises from 0.6% to 2% by weight of dehydroxanthan gum, based on a total weight of the hydrogel.

18. The cosmetic hydrogel of claim 15, wherein the hydrogel comprises a total of from 0.1% to 3% by weight of xanthan gum and/or carrageenan, based on a total weight of the hydrogel.

19. The cosmetic hydrogel of claim 15, wherein the hydrogel comprises a total of from 0.2% to 0.7% by weight of xanthan gum and/or carrageenan, based on a total weight of the hydrogel.

20. The cosmetic hydrogel of claim 15, wherein the hydrogel comprises from 0.1% to 3% by weight of xanthan gum, based on a total weight of the hydrogel.

21. The cosmetic hydrogel of claim 15, wherein the hydrogel comprises from 0.2% to 0.7% by weight of xanthan gum, based on a total weight of the hydrogel.

22. The cosmetic hydrogel of claim 15, wherein the hydrogel comprises a total of from 0.1% to 3% by weight of carrageenan, based on a total weight of the hydrogel.

23. The cosmetic hydrogel of claim 15, wherein the hydrogel comprises a total of from 0.2% to 0.7% by weight of carrageenan, based on a total weight of the hydrogel.

24. The cosmetic hydrogel of claim 15, wherein a ratio by weight of dehydroxanthan gum to a total of xanthan gum and/or carrageenan is from 10:1 to 1:1.

25. The cosmetic hydrogel of claim 15, wherein a ratio by weight of dehydroxanthan gum to a total of xanthan gum and/or carrageenan is from 5:1 to 2:1.

26. The cosmetic hydrogel of claim 15, wherein the hydrogel comprises at least 70% by weight of water, based on a total weight of the hydrogel.

27. The cosmetic hydrogel of claim 15, wherein the hydrogel comprises at least 77% by weight of water, based on a total weight of the hydrogel.

28. The cosmetic hydrogel of claim 15, wherein the hydrogel is free from polymers obtained by homo- or copolymerization with vinyl pyrrolidone, acrylic acid and/or methacrylic acid.

29. The cosmetic hydrogel of claim 15, wherein the hydrogel comprises one or more short-chain polyols having 2 to 8 carbon atoms.

30. The cosmetic hydrogel of claim 29, wherein the hydrogel comprises from 1% to 30% by weight of the one or more short-chain polyols having 2 to 8 carbon atoms, based on a total weight of the hydrogel.

31. The cosmetic hydrogel of claim 29, wherein the one or more short-chain polyols having 2 to 8 carbon atoms comprise one or more of glycerin, methylpropanediol, propylene glycol, hexane-1,2-diol, caprylyl glycol, butylene glycol, and pentylene glycol.

32. The cosmetic hydrogel of claim 31, wherein the one or more short-chain polyols having 2 to 8 carbon atoms comprise hexane-1,2-diol.

33. The cosmetic hydrogel of claim 32, wherein the hydrogel comprises from 0.2% to 2% by weight of hexane-1,2-diol, based on a total weight of the hydrogel.

34. A method of stabilizing the viscosity of a hydrogel which comprises dehydroxanthan gum and water, wherein the method comprises including in the hydrogel xanthan gum and/or carrageenan.
